(19) United States
(12) Patent Application Publication
(10) Pub. No.: US 2011/0082086 A1
(43) Pub. Date: Apr. 7, 2011

(54) MEDICAMENT FOR USE IN CONNECTION WITH CARTILAGE IMPAIRMENT

(75) Inventor: Stefan G. Pierzynowski, Malmo (SE)

(73) Assignee: Entress AB, Stockholm (SE)

(21) Appl. No.: 12/897,313

(22) Filed: Oct. 4, 2010

Related U.S. Application Data
(63) Continuation of application No. 12/084,947, filed on May 12, 2008, now abandoned.
(60) Provisional application No. 60/597,172, filed on Nov. 15, 2005.

(51) Int. Cl. A61K 38/05; A61K 31/194; A61K 31/198; A61K 31/401; A61K 38/06; A61P 19/04

(52) U.S. Cl. 514/17.1; 514/574; 514/561; 514/564; 514/423

(57) ABSTRACT

Use of a substance for treating joint medical conditions. The use includes the use of alpha-ketoglutarate acid.

### FIELD OF THE INVENTION

The present invention refers to medical compositions and uses of said compositions for the treatment, alleviation and prophylaxis of conditions associated with cartilage impairment and pain related to it, or prophylaxis of artrose and rheumatoid arthritis and pain related to it.

### BACKGROUND

As many as one out of three adults in the industrial world may currently suffer from chronic joint symptoms or arthritis. The most common symptom, persistant joint pain, can appear as hip pain, knee pain, hand pain or wrist pain, as well as joint pain in other areas of the body. The symptoms cause suffering and economic losses when persons are forced to stop working or cut down on working hours. Big sums are also spent on medical care. Obviously, there is a need for cost efficient solutions for alleviation or curing of joint symptoms and arthritis.

### SUMMARY OF THE INVENTION

Embodiments of the invention include the use of a substance including at least one member selected from the group consisting of alpha-ketoglutaric acid, glutamine, glutamic acid and pharmaceutically acceptable salts of these acids, amides of alpha-ketoglutaric acid and an amino acid or a di- or tripeptide dipeptides of glutamine and another amino acid, tripeptides of glutamine and other amino acids, dipeptides of glutamine acid and other amino acids, tripeptides of glutamic acid and other amino acids and pharmaceutically acceptable salts of said dipeptides and tripeptides, pharmaceutically accepted physical mixtures of alpha-ketoglutaric acid or a pharmaceutically acceptable salt thereof and at least one amino acid for the manufacture of a pharmaceutical preparation for the treatment or prophylaxis of a condition of inflammatory or non-inflammatory impairment of cartilage and pain related to above

Further embodiments include the use as stated above for the treatment or prophylaxis of artrose and rheumatoid arthritis and pain related to above.

Further embodiments includes the use as stated above for the treatment or prophylaxis of cartilage impairment at conditions involving weight loss and/or impaired nutrition, or gastrectomy, partial gastrectomy or gastric banding.

Further embodiments include the use as stated above for the treatment or prophylaxis of cartilage impairment at conditions involving malnutrition.

Further embodiments include the use as stated above for the relieving of pain associated with cartilage impairment at conditions mentioned above.

Further embodiments include the use as stated above for treatment or prophylaxis of osteoporosis related to gastrectomy.

### BRIEF DESCRIPTION OF THE DRAWINGS

The present invention will be further explained in the following description with the aid of preferred embodiments, example studies and accompanying drawings of which

FIG. 1 is a diagram describing the effect of dietary aplha-ketoglutarat and gastrectomy on body weights of rats;

FIG. 2 shows four transillumination photos of superior portion of cranium (calvaria) of experimental rats;

FIG. 3 is a diagram showing the area of lacunas as found on transillumination photos of calvaria of experimental rats.

### DETAILED DESCRIPTION

Thus according to one aspect of the present invention, there is provided the new use of at least one member selected from the group consisting of alpha-ketoglutaric acid, glutamine, glutamic acid and pharmaceutically acceptable salts of these acids, amides of alpha-ketoglutaric acid and an amino acid or a di- or tripeptide dipeptides of glutamine and another amino acid, tripeptides of glutamine and other amino acids, dipeptides of glutamine acid and other amino acids, tripeptides of glutamic acid and other amino acids and pharmaceutically acceptable salts of said dipeptides and tripeptides, pharmaceutically accepted physical mixtures of alpha-ketoglutaric acid or a pharmaceutically acceptable salt thereof and at least one amino acid for the manufacture of a pharmaceutical preparation for the treatment or prophylaxis of a condition of artrose, rheumatoid arthritis and cartilage destruction and pain related to above disorders.

According to a preferred embodiment of the invention alpha-ketoglutaric acid or an alkali or alkaline earth metal salt thereof or a combination thereof is used. Preferably sodium alpha-ketoglutarate is used.

According to another aspect of the present invention there is provided a method for the treatment method for the treatment or prophylaxis of a condition of increased pain of at least one member selected from the group consisting of artrose in mammals, including man, which method comprises administering to a subject in need for such treatment or prophylaxis of an effective pain amount of at least one member selected from the group consisting of alpha-ketoglutaric acid, glutamine, glutamic acid and pharmaceutically acceptable salts of these acids, amides of alpha-ketoglutaric acid and an amino acid or a di- or tripeptide, dipeptides of glutamine and another amino acid, tripeptides of glutamine and other amino acids, dipeptides of glutamic acid and other amino acids, tripeptides of glutamic acid and other amino acids and pharmaceutically acceptable salts of said dipeptides and tripeptides, pharmaceutically accepted physical mixtures of alpha-ketoglutaric acid or a pharmaceutically acceptable salt thereof and at least one amino acid.

According to preferred embodiments of these aspects alpha-ketoglutaric acid or an alkali or alkali or alkaline earth metal salt thereof or a combination thereof is administered. Most preferably sodium alpha-ketoglutarate is administered.

The pharmaceutical preparations of the active principle or principles used in accordance with the present invention may be administered to a vertebrate, including mammals and birds, such as rodent, such as a mouse, rat, guinea pig, or a rabbit; a bird, such as a turkey, hen or chicken and other broilers and free going animals; a cow, a horse, a pig or piglet and other farm animals, a dog, a cat and other pets, and in particular humans.

Administration may be performed in different ways depending what species of vertebrate to treat, on the condition of the vertebrate in the need of said methods, and the specific indication to treat.

In one embodiment, the administration is done as a food or feed supplement, such as a dietary supplement and/or a component in form of solid food and/or beverage. Further embodiments may be in suspensions or solutions, such as a beverage further described below. Also, the formats may be in capsules or tablets, such as chewable or soluble, e.g. effervescent tablets, as well as powder and other dry formats known to the skilled man in the art, such as pellets, such as micropellets, and grains.

The administration may be as a parenteral, rectal or oral food or feed supplement, as revealed above. Parenteral vehicles include sodium chloride solution, Ringer's dextrose, dextrose and sodium chloride, lactated Ringer's or fixed oils.

The food and feed supplement may also be emulsified. The active therapeutic ingredient or ingredients may then be mixed with excipients, which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH, buffering agents, which enhance the effectiveness of the active ingredient.

Different formats of the parental food or feed supplement may be supplied, such as solid food, liquids or lyophilized or otherwise dried formulations. It may include diluents of various buffers (e.g., Tris-HCl, acetate, phosphate), pH and ionic strength, additives such as albumin or gelatine to prevent absorption to surfaces, detergents (e.g., Tween 20, Tween 80, Pluronic F68, bile acid salts). solubilizing agents (e.g., glycerol, polyethyleneglycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the composition, complexation with metal ions, or incorporation of the material into or ontoparticulate preparations of polymeric compounds such as polylactic acid, polglycolic acid, hydrogels, etc, or onto liposomes, microemulsions, micelles, unilamellar or multilamellarvesicles, erythrocyte ghosts, or spheroplasts.

In one embodiment, the food or feed supplement is administered in the form of a beverage, or a dry composition thereof, in any of the methods according to the invention.

The beverage comprises an effective amount of the active ingredient or ingredients thereof, together with a nutritionally acceptable water-soluble carrier, such as minerals, vitamins, carbohydrates, fat and proteins. All of these components are supplied in a dried form if the beverage is provided in a dry form. A beverage provided ready for consumption further comprises water. The final beverage solution may also have a controlled tonicity and acidity, e.g. as a buffered solution according to the general suggestions in the paragraph above.

The pH is preferably in the range of about 2-5, and in particularly about 2-4, to prevent bacterial and fungal growth. A sterilised beverage may also be used, with a pH of about 6-8.

The beverage may be supplied alone or in combination with one or more therapeutically effective composition.

According to a further embodiment the pharmaceutical preparations as drug for oral and rectal use may be in the form of tablets, lozenges, capsules, powders, aqueous or oily suspensions, syrups, elixirs, aqueous solutions and the like comprising the active ingredient or ingredients in admixture with a pharmaceutically acceptable carrier and/or additives, such as diluents, preservatives, solubilizers, emulsifiers, adjuvants and/or carriers useful in the methods and use disclosed in the present invention.

Further, as used herein “pharmaceutically acceptable carriers” are well known to those skilled in the art and may include, but are not limited to 0.01-0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Amino acids forming part of amides with alpha-ketoglutaric acid or of dipeptides with glutamine or glutamic acid or tripeptides with glutamine and/or glutamic acid may be any of the amino acids occurring as components in peptides in nature. The same applies to the pharmaceutically accepted physical mixtures of alpha-ketoglutaric acid or salts thereof with at least one amino acid. Preferably the amino acid or acids is/are selected from the group consisting of arginine, omithine, leucine, isoleucine and lysine.

Said amino acids are preferably used in their L-configuration.

Example as of amides of alpha-ketoglutaric acid with an amino acid or a di- or tripeptide include, but are not limited to, amides of alpha-ketoglutaric acid with an amino acid selected from the group consisting of glutamine, glutamic acid, arginine, omithine, lysine, proline, isoleucine and leucine and amides of alpha-ketoglutaric acid with a dipeptide of glutamine and any of glutamic acid, arginine, omithine, lysine, proline, isoleucine and leucine and with a dipeptide of glutamic acid and any of arginine, ornithine, lysine, proline, isoleucine and leucine.

Examples of di- and peptides of glutamine and glutamic acid with other amino acids include those mentioned above in connection with amides of alpha-ketoglutaric acid with di- or tripeptides.

Examples of physical mixtures of a-ketoglutaric acid or salts thereof with at least one amino acid includes, but are not limited to physical mixtures of at least one member selected from the group consisting of alpha-ketoglutaric acid and the sodium, potassium, calcium and magnesium salts thereof with any of glutamine, glutamic acid, arginine, ornithine, leucine, isoleucine, lysine and proline and any combinations of said amino acids.

The molar ratio of alpha-ketoglutaric acid or salts thereof to amino acid or amino acids of said physical mixtures will in general be within the limits of from 1:0.01 to 1:2, preferably from 1:0.1 to 1:1.5 and most preferably from 1:0.2 to 1:1.0.

The dosage to be administered will vary depending on the active principle or principles to be used, the condition to be treated, the age, sex, weight etc. of the patient to be treated but will generally be within the range from 1 to 1000 mg/kg body weight/day, or from 10 to 400 mg/kg body weight and day, preferably from 10 to 100 mg/kg body weight/day.

The invention will now be further illustrated by means of example which should not be construed to limit the scope of the invention.

### Example

Background: Surgical removal of the stomach (gastrectomy, Gx) leads to osteoporosis in animals and in humans. Gastrectomy mainly affects the structure of trabecular bone. It is unclear whether Gx also adversely affects the epiphyseal plate. Dietary α-keto glutarate (AKG) is a precursor of hydroxyproline—the most abundant amino acid in the bone and cartilage pro-collagen. The aim of the studies was to highlight the effect of AKG on gastrectomy dependent bone/cartilage losses.

Methods: 40 female Sprague-Dawley rats were used. Twenty rats were gastrectomized and divided between 2 groups: Gx+AKG and Gx+Placebo. Another 20 rats were sham-operated and divided between another 2 groups: Sham+AKG and Sham+Placebo. After 8 weeks animals were sacrificed and calvarias, femora and tibiae were collected. Bone mass density (BMD) and bone mineral concentration (BMC) in right femora and tibiae were estimated and histomorphometry from left bones were estimated. Measurements of transillumination of calvarias were also performed.

Results: Dietary α-ketoglutarate revealed a strong protective effect on calvarias bone losses of gastrectomized rats. AKG exhibits a strong anti destructive effect on epiphyseal plate cells, trabecular bone volume and shape of the trabeculas of gastrectomized rats.

Conclusions: AKG minimizes bone and cartilage destruction developed after stomach resection in rats.

Surgical removal of the stomach leads to osteopenia and arthritis in humans, the rat and other experimental animals. Gastrectomy is associated with osteopenia in humans. Gastric dysfunctions may also contribute to the development of osteoporosis in the elderly. Hence most of the studies concerning bone disease deal with patients after gastric resection

Gastrectomy mainly affects trabecular bone and at times also cortical bone, inducing a pronounced effect of calvaria bone destruction. Reduction of cortical and trabecular bone mass after gastrectomy has been reported in both human sexes. Trabecular bone volume in tibia and femur is reduced by 60% after 16 weeks post-gastrectomy. Bone losses increase the risk of fractures of the hip, vertebrae and other sites among gastrectomy patients, which is a serious problem nowadays.

It is postulated that bone loss in gastrectomized patients is not a result of dietary deficiencies (e.g. calcium) or lack of gastric acid or Vitamin D. The mechanism behind the gastrectomy-evoked osteopenia is still unknown. It is postulated, however, that the primary cause of osteoporoses is inefficient re-syntheses of bone collagen after its massive destruction by osteoclasts. The main component of bone pro-collagen is proline—the amino acids synthesize in the gastrointestinal tract from AKG via glutamate and via proline which in turn is converted in bone pro-collagen to hydroxyproline in the presence of AKG, vitamin C and Fe2+. It was recently shown that AKG has been effective in preventing bone loss in ovariectomized rats, and denervated bones in turkey. In consideration of all of the above, the main aim of the studies was to investigate whether dietary AKG can prevent bone and cartilage losses in gastrectomized rats.

### Animals and Surgical Procedures

Forty female Sprague-Dawley rats, 10 weeks old (220-230 g), were housed in Macrolon® cages (2 rats in each cage) and given a diet of standard rat food pellets (Lactamin, Vadstena, Sweden) and vehicle or AKG ad libitum dissolved in water (Table 1). The study lasted for 8 weeks. Rats were weighed every week.

The rats drank between 25 and 50 millilitres each day. In principle, it may be assumed that rats drink between 10 and 20% of body weight.

The rats of the AKG group drank approximately 25 ml of AKG drink per day. In 25 ml of drink there is 0.36 g of AKG, which gives approximately 1 to 1.4 g of AKG per kg rat body weight and day. The rats in the placebo (control) group drank approximately 50 ml of placebo drink per day.

### Surgery

Twenty rats were gastrectomized and divided between 2 groups: Gx+AKG and Gx+Placebo (10 rats in each group). The glandular portion of the stomach (i.e. the acid-producing part, fundus and the pyloric antrum) was resected after which the non-glandular part (forestomach) was joined with the duodenum end-to-end. 20 rats were sham-operated and divided between 2 groups: Sham+AKG and Sham+Placebo (10 rats in each group). Sham-operation involved a midline abdominal incision, manipulation of the stomach and closure of the incision. Anesthesia was achieved by subcutaneous injection of Ketalar® (50 mg/kg; Parke-Davis, Morris Plains, N.J., U.S.A.) and Stresnil® (40 mg/kg; Janssen-Cilag Pharma, Vienna, Austria). Analgesia was achieved by subcutaneous injection of Temgesic® (0.18 mg/kg; Schering-Plough, Kenilworth, N.J., U.S.A). Treatment was commenced of Sham+Placebo and Gx+Placebo groups with vehicle while Sham+AKG and Gx+AKG were treated with AKG.

Gx rats were injected by the intramuscular route once every second week (beginning the first week after surgery) with 0.4 mg/kg of vitamin B₁₂ (Betolvex® 1 mg/ml, Dumex, Copenhagen, Denmark) to compensate for the loss of the intrinsic factor which is essential for the absorption of vitamin B₁₂ and 20 mg Fe³⁺/kg of ferric hydroxide poly maltose complex (Ferrum® 50 mg Fe³⁺mg/ml, Vifor (International) Inc., St. Gallen/Switzerland) as a supplement for the anticipated poor absorption of iron due to the loss of gastric acid. These supplementations were without effect on the body weight development of rats that had not undergone surgical procedures.

During the experiment 8 animals died. The final number of animals (n) was 7 in Sham+Placebo, 10 in Sham+AKG, 8 in Gx+Placebo and 7 in Gx+AKG group.

All rats were sacrificed by exsanguinations from the abdominal aorta under anaesthesia as mentioned above.

Studies were approved by the local Animal Welfare Committee, Lund, Sweden.

### Tissue Collection and Analysis

The calvaria were dissected out from each rat and cleaned of soft tissue by removing the periosteum carefully. Drying was avoided by covering each calvaria with gauze soaked in saline and storing them in an airtight container at +4° C. until examination. Each calvarium was placed on a glass plate on top of a light source (commercial fluorescent tube), emitting light of constant intensity. The resulting transillumination images were photographed by the use of a camera connected to an operation microscope, magnification ×16. The images were subjected to histomorphometric computer analysis carried out by Image J v. 1.33a. Percentage of bone lose (as observed area of lacunas) was estimated.

Both the femora and tibiae were collected and stored in 70% ethanol until further analysis.

Right femora and tibia were subjected to PIXIMUS® analysis, which gave the BMD in g/cm² and the BMC in g/cm³.

Ethanol fixed left femora and tibiae were decalcified in 7% nitrogen acid for 48 hours. Distal femur and proximal tibia specimens (consisted of epiphysis with 8 mm part of metaphysis) were used for further histological processes. The specimens were immersed in paraffin. Longitudinal sections of femur and tibia specimens (6 μm thick) were cut by automatic microtome Microm HM 360. Twenty slices (with 20 μm interval after each 5) per 1 bone from 1 individual were cut. Slices were stained with hematoxylin/eosin under standard conditions. Microscopic images were taken from each stained slice. The pictures used to evaluate trabecular bone were taken using a Nikon Eclipse E800—light microscope, magnification ×40 and Nikon D70—digital photo camera. The microscopic images of sections of femur and tibia were subjected to histomorphometric computer analysis. Trabeculas were analyzed using ImageJ v. 1.33a. The pictures used to evaluate epiphyseal plate were made by means of the Nomarski contrast technique and collected by AXIOVERT 200 M equipped with an LSM 5 Pascal laser scanning head, Zeiss, magnification ×100, with argon laser wave length 514 nm. Epiphyseal plate was analyzed using Analysis v. 3.0. Articular cartilage images were captured using fluorescent mode of AXIOVERT 200 M equipped with an LSM 5 Pascal laser scanning head, Zeiss, magnification ×100, with argon laser wave length 514 nm. Pictures of articular cartilage were evaluated by Zeiss LSM Image Examiner v. 3.1.0.99. Considered parameters with regard to trabeculas below epiphyseal plate were: trabecular bone volume (BV/TV %) measured to obtain characteristics of cancellous bone, and trabecular fractal dimensions (Box Counting Method). Parameters with regard to epiphyseal plate were: number of cartilage cells inside the ROI (Region Of Interest) consisting of Resting zone, Proliferative zone and Hypertrophic cartilage zone. Estimation of relative collagen content of the articular cartilage was made by eosin stained collagen fluorescence intensity measurement in random choice ROI (the same area for every slice—6 circles each 83 μm in diameter, along the articular cartilage), with LSM 5 Pascal laser scanning head detector 12 bit grey level as a scale of measurement. Measurements were taken in exactly the same standard conditions for every slice.

### Statistics

Data were compared with one way analysis of variance (ANOVA), Student's t-test, and p<0.05 was considered statistically significant.

### Results

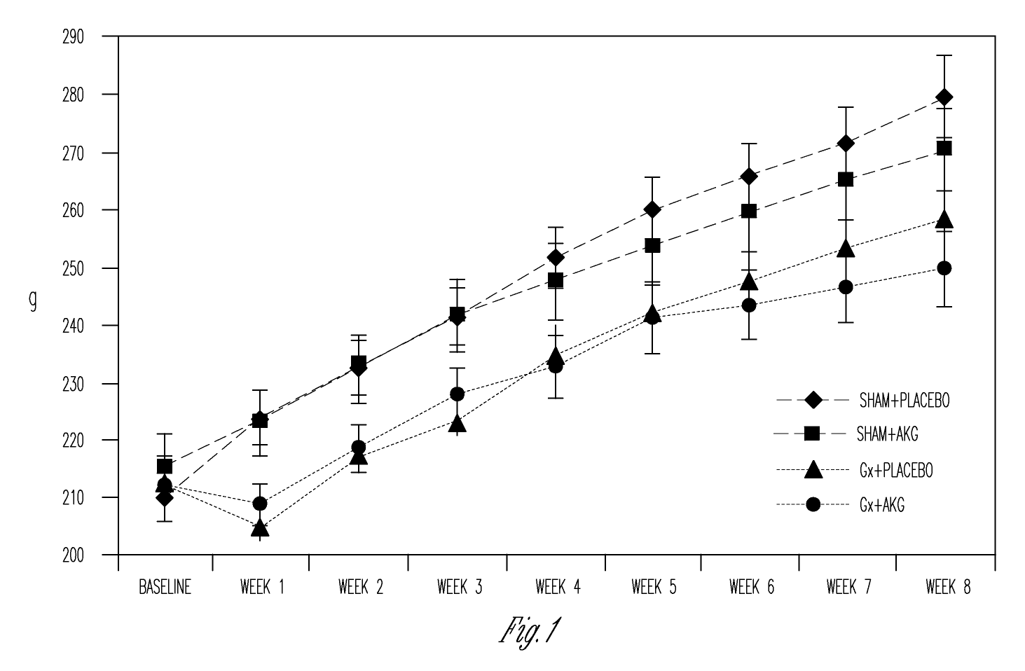

In the end of the experiment the body mass of surgically-treated animals was 8% less than sham-operated. There were no statistically significant differences between groups (FIG. 1.).

### Transillumination of Calvaria

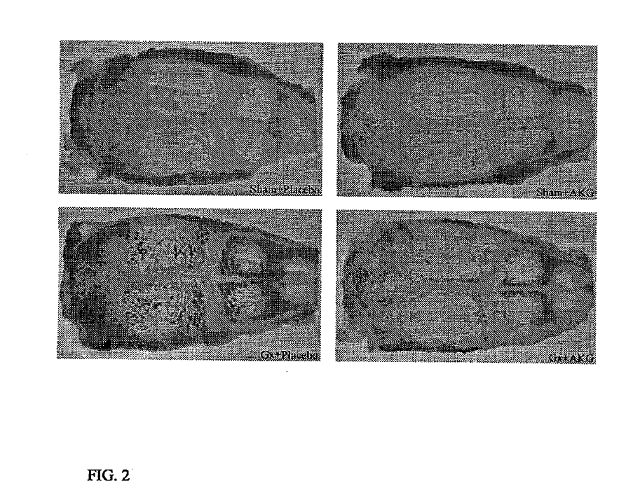

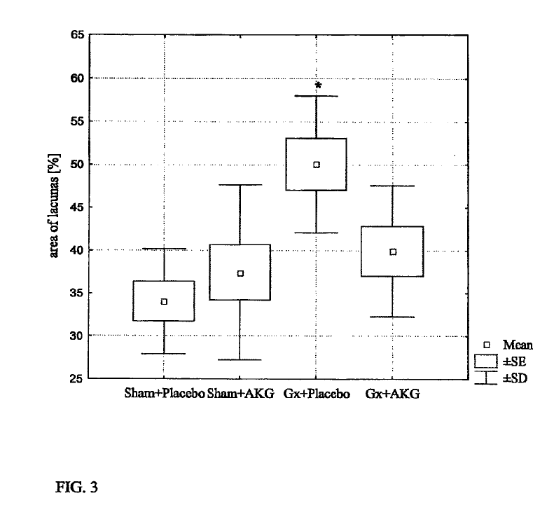

Transillumination of calvaria showed significant growth in percentage of bone lacunas in the Gx+Placebo and Gx+AKG rats compared to Sham+Placebo and Sham+AKG rats (FIG. 2.). Gx+AKG rats also exhibit a significantly lower percentage of lacunas compared to the Gx+Placebo group (*p=0.031) (FIG. 3.). The differences between Sham+Placebo and Sham+AKG were not statistically significant.

### Bone Mineral Density (BMD) and Bone Mineral Content (BMC) in Femur and Tibia

The BMD and BMC were lower in the Gx+Placebo and Gx+AKG rats as compared to Sham+Placebo and Sham+AKG rats (data not shown). However, BMD in Gx+AKG tended to be bigger than in Gx+Placebo group (p=0.19).

### Histomorphometry

### Articular Cartilage Analysis

The amount of cartilage collagen in Gx+AKG group was similar to that in control (sham-operated) groups and was significantly higher in comparison to Gx+Placebo group (Table 2.).

### Epiphyseal Plate Analysis

Quantitative estimation of epiphyseal growth plate cells showed an increase in the number of cells in group Gx+AKG (both in femur and tibia) compared to Gx+Placebo. Moreover the number of cartilage cells in Gx+AKG group was significantly larger than in both Sham groups (Table 3, 4.).

### Trabecular Bone Volume

The trabecular bone volume decreased in the Gx+Placebo and Gx+AKG rats compared to Sham+Placebo and Sham+AKG rats. However, the reduction of the area of trabeculas in Gx+AK was lower that in Gx+Placebo group (Table 5, 6.).

### Fractal Dimension of Bone Trabeculas

The fractal dimension in Gx+AKG was similar to control groups and was higher than in Gx+Placebo (Table 7, 8.).

### Discussion

The aim of the experiment was to evaluate the effect of dietary α-ketoglutarate on bone loss caused by gastrectomy. Data obtained confirm that hypothesis. Indeed dietary AKG prevented bone and cartilage losses in gastrectomized rats. Our results are in agreement with recent experiments showing that AKG prevents the development of osteoporosis in ovariectomized rats and post-menopausal women.

Gastrectomy caused cartilage collagen and cartilage cell loss in the Gx+Placebo but not Gx+AKG rats. 22% more cartilage cells were affirmed in Gx+AKG than in the Gx+Placebo group. This indicates that AKG was effective in preventing the loss of cartilage cells in the gastrectomized rats. Analysis revealed a protective effect of AKG on bone and cartilage collagen. The amount of collagen in the Gx+AKG group was within the range of the control groups for the experiment and was about 18% higher than in Gx+Placebo rats.

A protective effect of AKG on calvaria bone in gastrectomized rats was observed. Calvarias from Gx+AKG rats showed 20% less injury than those from Gx+Placebo rats. BMD and BMC values demonstrated that gastrectomy caused osteopenia in the Gx+Placebo and Gx+AKG rats which is in agreement with other experiments. However, using more sensitive histomorphometric methods we shown that AKG is possibly effective in preventing osteopenia in the GX rats.

Further, examined trabecular bone volume showed 38% less decrease in Gx+AKG rats compared to Gx+Placebo animals. Moreover, the fractal dimension of trabeculas in the Gx+AKG group showed almost the same level as in sham-operated groups. Thus α-ketoglutarate indeed has a strong influence on remodelling of structure of bone trabeculas.

Gastrectomy has a strong destructive effect on the skeleton, causing osteopenia and arthropathy. AKG cannot totally stop these injuries but it definitely limited profound destructive gastrectomy-related changes in bones and cartilage and probably improved remodelling of the skeletal system. The implications of these observations can be important for clinical consideration in humans e.g. where partial gastrectomy is recommended for weight loss in obese patients. All these patients develop osteoporoses and arthropathy. Thus, one can speculate that dietary AKG for these patients can stop or limit these destructive bone changes.

TABLE 1
Composition of AKG and placebo drinks.

| | AKG | Placebo |
|---|---|---|
| Ingredients | (g/dm³) | (g/dm³) |
| AKG (α-keto glutarate) | 14.6 | 0 |
| HCl hydrochloric acid | 0 | 3.32 |
| C₆H₁₂O₆ Glucose | 30.0 | 30.0 |
| C₁₂H₂₂O₁₁ Sucrose | 15.0 | 15.0 |
| NaOH sodium hydroxide | 3.6 | 3.6 |
| KOH potassium hydroxide | 0.75 | 0.75 |
| Ca(OH)₂ calcium hydroxide | 0.46 | 0.46 |
| Mg(OH)₂ magnesium | 0.18 | 0.18 |
| hydroxide | | |
| pH | 4.6 | 4.6 |

To achieve the same level of pH in each solution the Placebo drink was titrated with 0.1 M HCl to pH 4.6 (the pH-level of the AKG drink).

TABLE 2
Effect of AKG and gastrectomy on articular
cartilage collagen relative content.

| Treatment | fluorescence | SD |
|---|---|---|
| Gx + AKG | 2363 ^{a} | 623 |
| Gx + Placebo | 1928 ^{b} | 647 |
| Sham + AKG | 2475 ^{a} | 457 |
| Sham + Placebo | 2171 ^{a} | 374 |

A different letter given with a result in a column describes significant difference when p < 0.05
n = 7 in Gx + AKG, n = 8 in Gx + Placebo, n = 10 in Sham + AKG, n = 7 in Sham + Placebo

TABLE 3
Effect of AKG and gastrectomy on number of
femoris epiphyseal plate chondrocytes.

| | Number of | |
|---|---|---|
| Treatment | cells/mm² | SD |
| Gx + AKG | 2220^{a} | 490 |
| Gx + Placebo | 1760^{b} | 360 |
| Sham + AKG | 1890^{b} | 330 |
| Sham + Placebo | 1850^{b} | 220 |

A different letter given with a result in a column describes significant difference when p < 0.05
n = 7 in Gx + AKG, n = 8 in Gx + Placebo, n = 10 in Sham + AKG, n = 7 in Sham + Placebo

TABLE 4
Effect of AKG and gastrectomy on number
of tibias epiphyseal plate chondrocytes.

| | Number of | |
|---|---|---|
| Treatment | cells/mm² | SD |
| Gx + AKG | 2470^{a} | 470 |
| Gx + Placebo | 1950^{b} | 330 |
| Sham + AKG | 1840^{b} | 410 |
| Sham + Placebo | 2110^{b} | 340 |

A different letter given with a result in a column describes significant difference when p < 0.05
n = 7 in Gx + AKG, n = 8 in Gx + Placebo, n = 10 in Sham + AKG, n = 7 in Sham + Placebo

TABLE 5
Effect of α-ketoglutarate and gastrectomy
on femoris trabecular bone volume.

| | Area of | |
|---|---|---|
| Treatment | trabecules (%) | SD |
| Gx + AKG | 18.8^{a} | 3.7 |
| Gx + Placebo | 11.2^{b} | 2.1 |
| Sham + AKG | 25.5^{c} | 7.8 |
| Sham + Placebo | 24.5^{c} | 5.9 |

A different letter given with a result in a column describes significant difference when p < 0.05
n = 7 in Gx + AKG, n = 8 in Gx + Placebo, n = 10 in Sham + AKG, n = 7 in Sham + Placebo

TABLE 6
Effect of α-ketoglutarate and gastrectomy
on tibias trabecular bone volume.

| | Area of | |
|---|---|---|
| Treatment | trabeculas (%) | SD |
| Gx + AKG | 16.7^{a} | 3.4 |
| Gx + Placebo | 10.5^{b} | 2.5 |
| Sham + AKG | 24.9^{c} | 5.3 |
| Sham + Placebo | 21.1^{c} | 5.7 |

A different letter given with a result in a column describes significant difference when p < 0.05
n = 7 in Gx + AKG, n = 8 in Gx + Placebo, n = 10 in Sham + AKG, n = 7 in Sham + Placebo

TABLE 7
Effect of α-ketoglutarate and gastrectomy
on fractal dimension of femoris trabeculas.

| | Fractal | |
|---|---|---|
| Treatment | dimension [D] | SD |
| Gx + AKG | 1.22^{b} | 0.02 |
| Gx + Placebo | 1.19^{a} | 0.03 |
| Sham + AKG | 1.24^{b} | 0.04 |
| Sham + Placebo | 1.25^{b} | 0.03 |

A different letter given with a result in a column describes significant difference when p < 0.05
n = 7 in Gx + AKG, n = 8 in Gx + Placebo, n = 10 in Sham + AKG, n = 7 in Sham + Placebo

TABLE 8
Effect of α-ketoglutarate and gastrectomy
on fractal dimension of tibias trabeculas.

| | Fractal | |
|---|---|---|
| Treatment | dimension [D] | SD |
| Gx + AKG | 1.22^{b} | 0.02 |
| Gx + Placebo | 1.17^{a} | 0.02 |
| Sham + AKG | 1.22^{b} | 0.03 |
| Sham + Placebo | 1.21^{b} | 0.04 |

A different letter given with a result in a column describes significant difference when p < 0.05
n = 7 in Gx + AKG, n = 8 in Gx + Placebo, n = 10 in Sham + AKG, n = 7 in Sham + Placebo

### LEGEND TO THE FIGURES

FIG. 1. Effect of dietary a-ketoglutarate and gastrectomy on body weights of rats. Control groups: SHAM+PLAC, GX+PLAC Experimental groups: SHAM+AKG, GX+AKG (SHAM—sham-operated rats, GX—gastrectomized rats).

FIG. 2. Selected photos of calvaria of tested animals. Control groups: SHAM+PLAC, GX+PLAC Experimental groups: SHAM+AKG, GX+AKG (SHAM—sham-operated rats, GX—gastrectomized rats).

FIG. 3. Effect of dietary α-ketoglutarate and gastrectomy on transillumination of calvaria. Control groups: SHAM+PLAC, GX+PLAC Experimental groups: SHAM+AKG, GX+AKG (SHAM—sham operated rats, GX—gastrectomized rats). *p=0.0288

1-12. (canceled)

13. A method for treatment of cartilage impairment comprising administering an effective amount of alpha-ketoglutaric acid or a pharmaceutically acceptable salt of alpha-ketoglutaric acid to a subject in need thereof.

14. The method according to claim 13, wherein the alpha-ketoglutaric acid or the pharmaceutically acceptable salt of alpha-ketoglutaric acid is in a pharmaceutically accepted physical mixture with at least one amino acid.

15. The method according to claim 14, wherein the amino-acid is selected from the groups consisting of glutamine, glutamic acid, arginine, ornithine, leucine, isoleucine, lysine, proline, and combinations thereof.

16. The method according to claim 13, wherein the pharmaceutically acceptable salt of alpha-ketoglutaric acid is an alkali salt or an alkaline earth metal salt or a combination thereof.

17. The method according to claim 16, wherein the pharmaceutically acceptable salt of alpha-ketoglutaric acid is sodium alpha-ketoglutarate.

18. (canceled)

19. The method according to claim 13, where the dosage given to a subject is in the interval from 1 to 1000 mg/kg body weight/day of the substance.

20. The method according to claim 19, where the dosage given to a subject is in the interval from 10 to 400 mg/kg body weight/day of the substance.

21. The method according to claim 20, where the dosage given a subject is in the interval from 10 to 100 mg/kg body weight/day of the substance.

22. A method for treatment of cartilage impairment comprising administering an effective amount of an amide of alpha-ketoglutaric acid and an amino acid, a dipeptide or a tripeptide, to a subject in need thereof.

23. The method according to claim 22, where the amino-acid which forms the amide with alpha-ketoglutaric acid is selected from: glutamine, glutamic acid, arginine, ornithine, lysine, proline, isoleucine and leucine.

24. A method according to claim 22, where the dipeptide which forms the amide with alpha-ketoglutaric acid is a dipeptide of glutamine and any of: glutamic acid, arginine, ornithine, lysine, proline, isoleucine and leucine.

25. (canceled)

26. The method according to claims 22, where the dosage given to a subject is in the interval from 1 to 1000 mg/kg body weight/day of the substance.

27. The method according to claims 26, where the dosage given to a subject is in the interval from 10 to 400 mg/kg body weight/day of the substance.

28. The method according to claims 27, where the dosage given to a subject is in the interval from 10 to 100 mg/kg body weight/day of the substance.

29. A method for prophylaxis of cartilage impairment comprising administering an effective amount of alpha-ketoglutaric acid or a pharmaceutically acceptable salt of alpha-ketoglutaric acid to a subject in need thereof.

30. The method according to claim 29, wherein the alpha-ketoglutaric acid or the pharmaceutically acceptable salt of alpha-ketoglutaric acid is in a pharmaceutically accepted physical mixture with at least one amino acid.

31. The method according to claim 30, wherein the amino-acid is selected from the groups consisting of glutamine, glutamic acid, arginine, ornithine, leucine, isoleucine, lysine, proline, and combinations thereof.

32. The method according to claim 29, wherein the pharmaceutically acceptable salt of alpha-ketoglutaric acid is an alkali salt or an alkaline earth metal salt or a combination thereof.

33. The method according to claim 32, wherein the pharmaceutically acceptable salt of alpha-ketoglutaric acid is sodium alpha-ketoglutarate.

34. The method according to claim 29, where the dosage given to a subject is in the interval from 1 to 1000 mg/kg body weight/day of the substance.

35. The method according to claim 34, where the dosage given to a subject is in the interval from 10 to 400 mg/kg body weight/day of the substance.

36. The method according to claim 35, where the dosage given a subject is in the interval from 10 to 100 mg/kg body weight/day of the substance.

37. A method for prophylaxis of cartilage impairment comprising administering an effective amount of an amide of alpha-ketoglutaric acid and an amino acid, a dipeptide or a tripeptide, to a subject in need thereof.

38. The method according to claim 37, where the amino-acid which forms the amide with alpha-ketoglutaric acid is selected from: glutamine, glutamic acid, arginine, ornithine, lysine, proline, isoleucine and leucine.

39. A method according to claim 37, where the dipeptide which forms the amide with alpha-ketoglutaric acid is a dipeptide of glutamine and any of: glutamic acid, arginine, ornithine, lysine, proline, isoleucine and leucine.

40. The method according to claims 37, where the dosage given to a subject is in the interval from 1 to 1000 mg/kg body weight/day of the substance.

41. The method according to claims 40, where the dosage given to a subject is in the interval from 10 to 400 mg/kg body weight/day of the substance.

42. The method according to claims 41, where the dosage given to a subject is in the interval from 10 to 100 mg/kg body weight/day of the substance.
